(19) United States
(12) Patent Application Publication
(10) Pub. No.: US 2026/0263008 A1
(43) Pub. Date: Sep. 10, 2026

(54) TOILET SEAT AND INTELLIGENT TOILET

(71) Applicant: Fortune Brands Innovations (Shanghai) Co. Ltd., Shanghai (CN)

(72) Inventors: Chenjie YU, Shanghai (CN); Jian JIANG, Shanghai (CN); Haijiang YI, Shanghai (CN); Chun CHEN, Shanghai (CN)

(21) Appl. No.: 19/073,683

(22) Filed: Mar. 7, 2025

(51) Int. Cl. A61B 5/00; A47K 13/24; A61B 5/0537

(52) U.S. Cl. CPC A61B 5/6891; A47K 13/24; A61B 5/0537; A61B 5/4872

(57) ABSTRACT

The present disclosure relates to the field of intelligent toilets and discloses a toilet seat and an intelligent toilet. The toilet seat comprises a toilet seat body and a connecting mechanism, wherein a rear end of the toilet seat body is rotatably connected to a surface of a toilet seat stand via the connecting mechanism; the toilet seat body comprises a seat face plate, a seat bottom plate and health detection sensors mounted on the seat face plate; the connecting mechanism is connected in a way that a gap is formed between the connecting mechanism and the toilet seat body, so as to allow the toilet seat body to move vertically with respect to the connecting mechanism and be supported on a top surface of the toilet seat by at least some of the health detection sensors when a human body is seated on the seat face plate.

### FIELD

The present disclosure relates to the field of intelligent toilets, in particular to a toilet seat capable of health detection and an intelligent toilet having same.

### BACKGROUND

Intelligent toilets are usually used for medical treatment and health care for the elderly. They were initially designed with a warm water washing function, and provided with a variety of additional functions later, such as heating with the toilet seat cover, washing with warm water, drying with warm air, and sterilization, etc. Generally, there are three types of intelligent toilets available in the market, i.e., intelligent toilets with washing, heating and sterilization functions, intelligent toilets with an automatic toilet seat cover replacement function, and intelligent toilets with automatic toilet seat cover replacement and washing functions.

In recent years, people are subject to increasingly higher work and life stresses. As a result, most people are in a sub-health status. However, people in a sub-health status are often unaware of their physical changes, and they go to the hospital only when they feel uncomfortable and can't relieve the uncomfortableness by themselves, but, at that point, their conditions may have developed to a very serious level. Consequently, not only the best opportunity for treatment is lost, but also the pain and economic burden are increased. For that reason, more and more users hope that intelligent toilets can collect human health information, so that the users can learn about their health status during the toilet time. Among many parameters indicating human physical health, weight is an important parameter.

In addition, with the continuous development of science and technology, people's understanding on health is becoming more and more profound. They want to accurately measure the content of human body fat and other substances, and obtain data of fat, moisture and proteins in human body. At present, there are mainly two types of sensors for measuring body fat in the market, which are made of ITO films and electrode plates respectively. It is found in research that when an electric signal having a specific frequency passes through human body, the “impedance” value of the fat part is higher than that of muscles and other tissues of the human body. Thus, the data related to body fat in human body can be obtained under a principle that a safe electrical signal having a specific frequency varies differently in different body tissues when it passes through human body owing to different “impedances” of different body tissues.

The detection of body weight and body fat has an important reference and guiding significance for comprehensive judgment on human health made in combination with other parameters. Therefore, it is desired that intelligent toilets may be capable of detecting body weight and body fat.

However, there is no intelligent toilet technology that has a body fat detection function in the prior art, let alone intelligent toilet technology capable of detecting body weight and body fat simultaneously. Intelligent toilets having a body weight detection function in the prior art usually have a complex structure, and have a toilet seat in a split design and in great thickness. Such intelligent toilets provide a poor user experience, have parts that are prong to fail, weak sealing performance, and a short service life.

In addition, in the prior art, the rear end of a toilet seat having a weight detection function is connected to a hinge shaft fixed on the toilet, and there is certain stress between them. As a result, the pressure on the body weight detection sensor is affected, the body weight can't be obtained correctly, and the accuracy of human body weight detection is compromised.

### SUMMARY

An object of the present disclosure is to provide a toilet seat having a health detection function and an intelligent toilet having the toilet seat, so as to solve the technical problems that the intelligent toilets capable of detecting body parameters in the prior art have a complex structure and can't detect body weight accurately.

To solve the technical problem described above, the present invention provides the following technical solutions.

A toilet seat, comprising a toilet seat body and a connecting mechanism for rotatably connecting the toilet seat body to a toilet seat stand, wherein the toilet seat body comprises a seat face plate, a seat bottom plate and health detection sensors mounted on the seat face plate, wherein the seat face plate and the seat bottom plate are snap-fitted with each other to form a cavity between the seat face plate and the seat bottom plate, and the health detection sensors are mounted in a way that a sensing terminal of each health detection sensor can sense a human body seated on the seat face plate directly and/or indirectly via the seat face plate, and output a sensing signal via a signal output terminal arranged inside the cavity; the connecting mechanism is connected to the toilet seat body in a way that a gap is formed between the connecting mechanism and the toilet seat body, so as to allow the toilet seat body to move vertically with respect to the connecting mechanism and be supported on a top surface of the toilet seat stand by at least some of the health detection sensors when the human body is seated on the seat face plate.

The working principle of the present disclosure is as follows: To use the toilet seat, a user may rotate the toilet seat body to the surface of the toilet seat stand via the connecting mechanism, now, the toilet seat body is in an initial state (position of use); then, the user sits on the surface of the toilet seat body, thereby the body of the user directly or indirectly acts on the sensing terminals of the health detection sensors; thus, the health detection sensors mounted on the toilet seat body can automatically detect the body health indicators of the user and collect relevant data. Since there is a gap between the connecting mechanism and the toilet seat body, the toilet seat body can be moved vertically with respect to the connecting mechanism when the body of the user is seated on the seat face plate, so that the measurement of the body weight carried out by the health detection sensors, such as pressure sensors, will not be significantly affected.

Preferably, the health detection sensors comprise pressure sensors, wherein a sensing terminal of each pressure sensor is fixed to an inner wall of the seat face plate, so that the pressure sensors can receive the pressure transferred by the seat face plate; via-holes are formed in the seat bottom plate, and a terminal of each pressure sensor away from the sensing terminal extends beyond the lower surface of the seat bottom plate through a via-hole, so that the pressure sensors can be supported on the top surface of the toilet seat during use; alternatively, the pressure sensors are mounted to the seat bottom plate, and a terminal of each pressure sensor away from the seat face plate extends beyond the lower surface of the seat bottom plate, so that the pressure sensors can be supported on the top surface of the toilet seat during use.

Preferably, the health detection sensors comprise a plurality of pressure sensors symmetrically distributed on left and right sides with respect to center lines of the seat face plate and the seat bottom plate.

Preferably, the health detection sensors comprise body fat sensors, and a sensing terminal of each body fat sensors is exposed on the upper surface of the seat face plate so that it can contact with the human body seated on the seat face plate.

Preferably, the upper surface of the seat face plate is provided with a plurality of grooves, and the health detection sensors comprise a plurality of body fat sensors respectively mounted in the grooves; the grooves are provided with through-holes at the bottom, and a signal output terminal of each body fat sensor is led through a through-hole.

Preferably, the connecting mechanism comprises a first connecting part and a second connecting part that are respectively used for connecting to two sides of the rear end of the toilet seat body, and both the first connecting part and the second connecting part are provided with support legs at the bottom, and the support legs are supported on the top surface of the toilet seat stand when the toilet seat body is rotated to a position of use.

Preferably, the first connecting part and the second connecting part are respectively provided with a connecting head for connecting to the toilet seat body and a pivoting part for connecting to the toilet seat stand, and a mating part corresponding to the rear end of the toilet seat body is formed on the outer periphery of the pivoting part, and the mating part is spaced from the rear end of the toilet seat body when the toilet seat body is rotated to the position of use.

Preferably, a bushing hole for leads to pass there-through is formed in the connecting head, and the leads are electrically connected to the signal output terminal of each health detection sensor.

Preferably, the seat face plate is further provided with a human body sensing sensor, and a sensing terminal of the human body sensing sensor is exposed on the upper surface of the seat face plate.

In another aspect, the present disclosure further provides an intelligent toilet, which comprises a toilet seat stand and the toilet seat described above, wherein the toilet seat is rotatably connected to the top surface of the toilet seat stand via the connecting mechanism.

The present disclosure further discloses a detection control method for a toilet seat capable of health detection, which utilizes the toilet seat capable of health detection described above, and comprises the following steps:
step 1: driving the connecting mechanism to rotate the toilet seat body to the surface of the toilet seat stand;
step 2: activating the health detection sensors on the toilet seat body;
step 3: collecting data of human health parameters, by the health detection sensors, when the health detection sensors detect that a human body is seated on the surface of the toilet seat body and has been in contact with the health detection sensors for a preset duration;
step 4: transmitting the collected data of human health parameters to an operating terminal after the data of human health parameters is collected, thereby completing the collection of human health parameters.

Preferably, in the step 2, the health detection sensors comprise body fat sensors and pressure sensors, wherein the body fat sensors are used for detecting body fat parameters, and the pressure sensors are used for detecting body weight.

Preferably, in the step 2, a human body sensing sensor is further provided on the surface of the toilet seat body, and the human body sensing sensor can wake up the pressure sensors and the body fat sensors only when it senses a human body seated on the toilet seat body.

The present disclosure has the following beneficial effects: The toilet seat capable of health detection in the present disclosure has the advantages of a simple overall structure, an integrated design, small overall size, convenient installation, a robust structural connection, stable and reliable operation during use, an attractive appearance, and a sanitary internal structure, etc.; the toilet seat capable of health detection in a preferred technical scheme of the present disclosure can detect a variety of physical health indicating parameters, realizes integration of multiple functions, simplifies operation steps, reduces detection difficulties, has higher detection precision and accuracy, and has a promising market value; the toilet seat has high measurement accuracy, since the connecting mechanism at the rear end of the body of the toilet seat capable of health detection is separate from the toilet seat and has no impact on the deformation of the toilet seat during use.

### BRIEF DESCRIPTION OF THE DRAWINGS

In order to make the object, technical scheme and advantages of the present disclosure understood more clearly, the present disclosure will be further described below in detail with reference to the accompanying drawings, in which:

FIG. 1 is an isometric view of the structure of the toilet seat in the present disclosure;

FIG. 2 is a top view of the structure of the toilet seat in the present disclosure;

FIG. 3 is a schematic structural diagram of the seat bottom plate in the present disclosure;

FIG. 4 is a schematic diagram of the internal structure of the toilet seat in the present disclosure;

FIG. 5 is an enlarged view of an area E in FIG. 4 in the present disclosure;

FIG. 6 is an exploded view of the structure of the pressure sensor in the present disclosure;

FIG. 7 is a schematic structural diagram of the connecting mechanism in the present disclosure;

FIG. 8 is a schematic structural diagram of the section A-A in FIG. 2 in the present disclosure;

FIG. 9 is a schematic structural diagram of the section B-B in FIG. 2 in the present disclosure;

FIG. 10 is a structural installation diagram of the pressure sensor on the seat bottom plate in another preferred embodiment of the present disclosure; and

FIG. 11 is an exploded view of the installation structure in FIG. 10.

Reference Numerals: 1—seat face plate; 11—groove; 2—seat bottom plate; 21—limiting groove; 22—via-hole; 31—first connecting part; 32—second connecting part; 33—bushing hole; 34—support leg; 35—mating part; 36—cogged slot; 37—protrusion; 41—pressure sensor; 411—first pressure sensing terminal; 412—force transfer part; 413—second pressure sensing terminal; 42—body fat sensor; 43—human body sensing sensor.

### DETAILED DESCRIPTION

To make the object, technical scheme, and advantages of the embodiments of the present disclosure understood more clearly, hereunder the technical scheme in the embodiments of the present disclosure will be detailed clearly and completely, with reference to the accompanying drawings in the embodiments of the present disclosure.

It may be noted that similar numbers and letters represent similar items in the following drawings. Therefore, once an item is defined in one of the drawings, it is not necessary to define and explain it further in the follow-up drawings. In the description of the present disclosure, it may be noted that the orientational or positional relations indicated by terms such as “central”, “upper”, “lower”, “left”, “right”, “vertical”, “horizontal”, “inside” or “outside”, etc., are based on the orientational or positional relations indicated in the accompanying drawings, or based on the orientational or positional relations in which the product in the present disclosure is usually placed. Those terms are intended only to ease and simplify the description of the present disclosure, but are not intended to indicate or imply that the involved device or element may have a specific orientation or may be constructed and operated in a specific orientation. Therefore, the use of those terms shall not be deemed as constituting any limitation on the present disclosure. Besides, the terms “first”, “second”, and “third”, etc., are only for a differentiation purpose, and may not be comprehended as indicating or implying any relative importance. In addition, the terms “horizontal” and “vertical”, and the like do not mean that the components may be absolutely horizontal or vertical; instead, the components may be slightly inclined. For example, “horizontal” only means that the direction is more horizontal than “vertical”, but doesn't mean that the structure is absolutely horizontal, which is to say, the structure may be slightly inclined. It may also be noted: in the description of the present disclosure, unless otherwise specified and defined explicitly, the terms “provide”, “install”, “interconnect”, and “connect”, etc. shall be interpreted in their general meanings, for example, a connection may be a fixed connection, detachable connection, or integral connection; may be a mechanical connection or electrical connection; or may be a direct connection or indirect connection via an intermediate medium, or internal communication between two elements. Those having ordinary skills in the prior art can understand the specific meanings of the terms in the present disclosure in their contexts.

The present disclosure solves the technical problems that the intelligent toilets capable of detecting body parameters in the prior art have complex structures and can only detect some specific parameters.

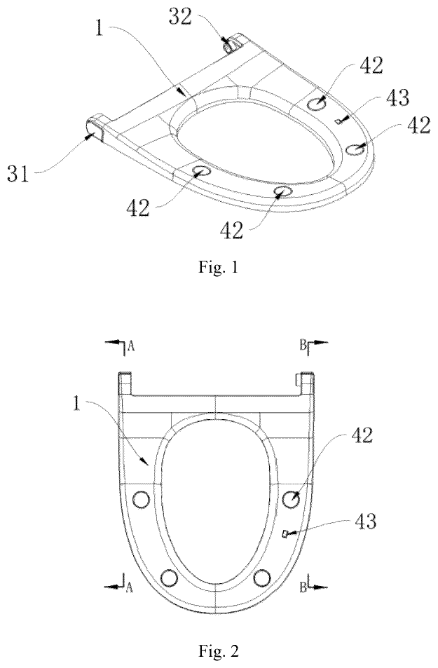

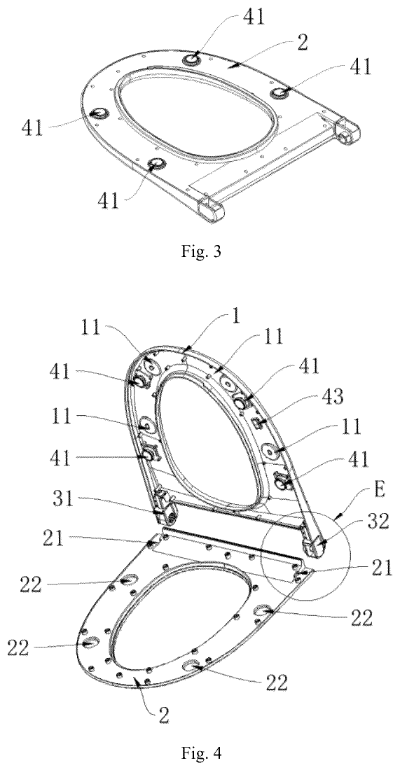

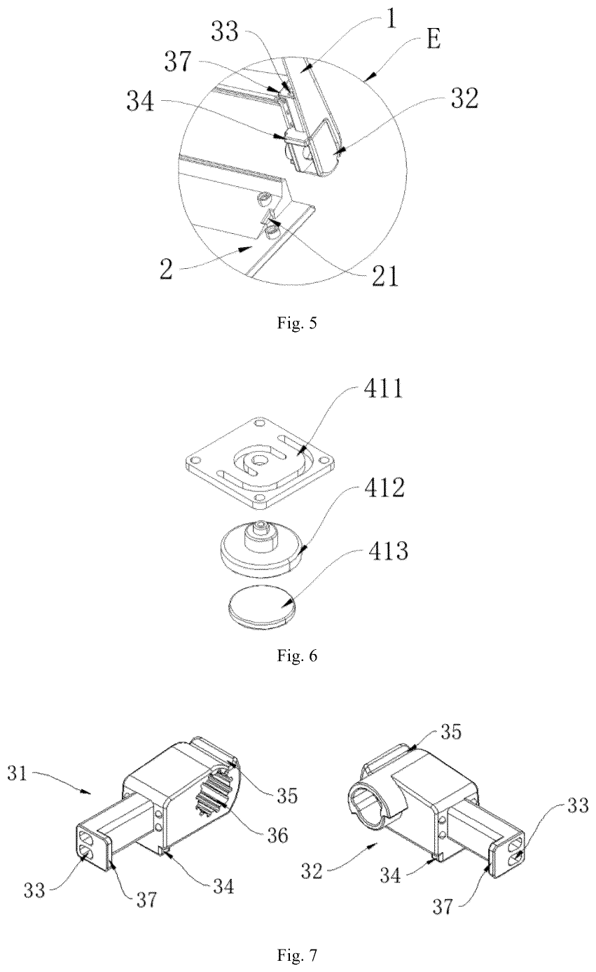

As shown in FIGS. 1 to 5, based on the above-mentioned technical problems to be solved, the present disclosure discloses a toilet seat capable of health detection, which is to be mounted on the surface of a toilet seat stand, and comprises a toilet seat body and a connecting mechanism, wherein a rear end of the toilet seat body is rotatably connected to the surface of the toilet seat stand via the connecting mechanism;
the toilet seat body comprises a seat face plate 1, a seat bottom plate 2 and health detection sensors mounted on the seat face plate 1; the seat face plate 1 and the seat bottom plate 2 are snap-fitted with each other to form a cavity between them, a sensing terminal of each health detection sensor is located on the surface of the seat face plate 1, and a signal output terminal of each health detection sensor is located in the cavity;
the connecting mechanism comprises a first connecting part 31 and a second connecting part 32, which are respectively snap-fitted with two sides of the rear end of the toilet seat body; there are gaps between the upper and lower surfaces of the first connecting part 31 and the second connecting part 32 and the inner surfaces of the seat face plate 1 and the seat bottom plate 2.

The working principle of the present disclosure is as follows: To use the toilet seat, the toilet seat body is driven by the connecting mechanism to rotate to the surface of the toilet seat stand, now, the toilet seat body is in an initial state; then, the user sits on the surface of the toilet seat body, thereby the body skin of the user comes into contact with the health detection sensors; thus, the health detection sensors mounted on the toilet seat body can automatically detect the health indicators of the body of the user and collect relevant data. Since there are gaps between the first connecting part 31 and the second connecting part 32 and the seat face plate 1 and seat bottom plate 2 of the toilet seat body, the toilet seat body can be moved vertically with respect to the connecting mechanism when the body of the user is seated on the seat face plate, so that the measurement of the body weight carried out by the health detection sensors, such as pressure sensors, will not be significantly affected.

Specifically, in actual use, a health detection controller is arranged between the first connecting part 31 and the second connecting part 32 for controlling the first connecting part 31 and the second connecting part 32 to rotate, so as to drive the toilet seat body to rotate, thereby the toilet seat body can be placed on the surface of the toilet seat stand or moved away from the surface of the toilet seat stand.

Preferably, as shown in FIG. 4, the health detection sensors comprise body fat sensors 42 and pressure sensors 41; the surface of the seat face plate 1 is provided with a plurality of grooves 11, a signal input terminal of each body fat sensor 42 is placed in a groove 11, each groove 11 is provided with a through-hole at the bottom, and a signal output terminal of each body fat sensor 42 is led through a through-hole; the pressure sensors 41 are fixed to the inner wall of the seat face plate 1.

The pressure sensors 41 are used to detect the weight of a human body and collect relevant data. During weight detection, when a human body sits on the toilet seat, the weight of the human body exerts pressure on the pressure sensors 41, and the pressure sensors 41 can identify and detect the sitting pressure when the human body sits on the toilet seat, so as to obtain body weight parameters of the human body. The body fat sensors 42 are used to detect the body fat rate of the human body and collect relevant data. To measure body fat data, the human body sits on the toilet seat and the skin of the human body contacts with all the body fat sensors 42; the body fat sensors 42 release micro-current so that a closed AC circuit is formed between the human body and the body fat sensors 42, thus an electrical impedance value is obtained; then, the body fat rate is calculated from the electrical impedance value in combination with other data such as body height and body weight.

After body fat data of the human body is collected by means of the signal input terminals of the body fat sensors 42, relevant parameters of the human body, such as water content, muscle content, bone content, protein content, visceral fat content, basal metabolism, age, etc., can be further calculated according to the body fat rate, body height and body weight.

In addition, water invading into the cavity can flow out of the cavity via the via-holes 22 in the seat bottom plate 2, thereby water accumulation in the cavity is avoided.

Preferably, an even number of pressure sensors 41 are provided, and the pressure sensors 41 are grouped into two groups, which are symmetrically distributed on the inner wall of the seat face plate 1 on left and right sides with respect to the center line of the toilet seat body, and the pressure sensors 41 in each group are evenly distributed.

Preferably, an even number of body fat sensors 42 are provided, and the body fat sensors are grouped into two groups, which are symmetrically distributed on the surface of the seat face plate 1 on left and right sides with respect to the center line of the toilet seat, and the body fat sensors 42 in each group are arranged on the surface of the seat face plate 1 where the human body can come in contact with the body fat sensors 42.

Preferably, as shown in FIG. 6, the pressure sensor 41 comprises a first pressure sensing terminal 411, a force transfer part 412 and a second pressure sensing terminal 413. As shown in FIG. 4, the first pressure sensing terminal 411 is fixedly connected to the inner wall of the seat face plate 1; the seat bottom plate 2 is provided with a plurality of via-holes 22 corresponding to the pressure sensors 41, and the force transfer part 412 runs through a via-hole 22 so that the sensing terminal of the second pressure sensing terminal 413 is exposed outside the lower surface of the seat bottom plate 2; the first pressure sensing terminal 411 is provided with a sensing hole, the top of the force transfer part 412 is movably connected to the first pressure sensing terminal 411 through the sensing hole, and the second pressure sensing terminal 413 is in interference fit with the bottom of the force transfer part 412.

Specifically, when there is no external force that exerts pressure on the toilet seat, the sensing terminal of the second pressure sensing terminal 413 is in contact with the toilet seat; the first pressure sensing terminal 411 of the pressure sensor 41 produces a downward pressure on the force transfer part 412 once it is subjected to a seating pressure, and the force transfer part 412 transfers the force to the second pressure sensing terminal 413, so that the downward pressure directly acts on the toilet seat stand; then the second pressure sensing terminal 413 provides an upward supporting force, the force transfer part 412 is supported by the supporting force from the second sensing terminal, thereby a relative movement occurs between the force transfer part 412 and the first pressure sensing terminal 411, and the pressure is sensed.

With such a structure, the toilet seat stand provides all supporting force (equal to the seating pressure but reversed in direction), thus, the downward pressure of the second pressure sensing terminal 413 will not be dispersed and transferred to other parts of the seat bottom plate 2. As a result, the seat bottom plate 2 will not be twisted and deformed and come into contact with the toilet face plate 1 locally and disperse the sitting pressure owing to the downward pressure, and the downward pressure will not be dispersed to the connecting structure that connects the toilet seat to the toilet at the rear end. Thus, all the sitting pressure and the supporting force are directly transferred and exerted on the first pressure sensing terminal 411 and the second pressure sensing terminal 413 of each pressure sensor 41, so that each pressure sensor 41 can sense the current change between the force transfer part 412 and the first pressure sensing terminal 411, thereby can accurately measure the downward sitting pressure produced when the human body sits on the toilet seat. In that way, highly accurate body weight detection is ensured, and the problem that the accuracy of body weight detection performed by the intelligent toilet is affected by the stress of the toilet seat connecting structure is avoided.

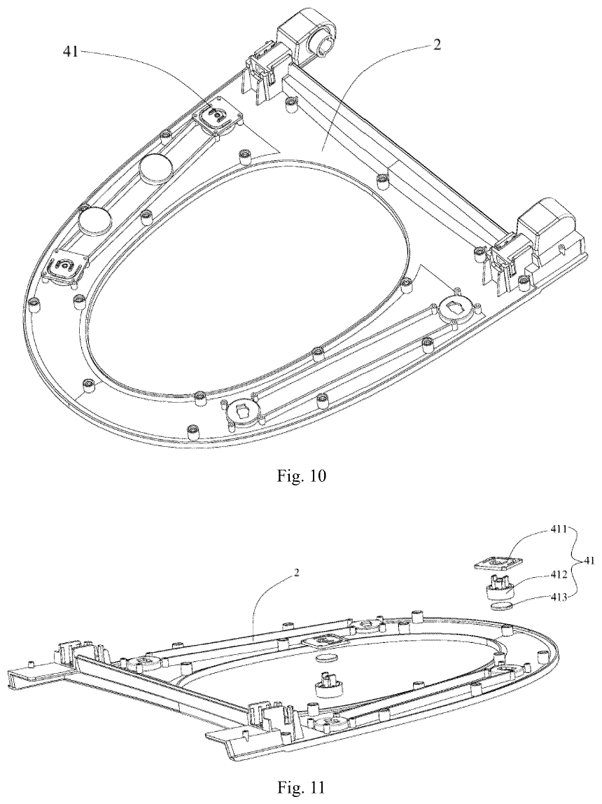

In another preferred embodiment shown in FIGS. 10 and 11, the pressure sensor 41 is mounted to the seat bottom plate 2 by fasteners, such as bolts. Likewise, the pressure sensor 41 may comprise a first pressure sensing terminal 411, a force transfer part 412 and a second pressure sensing terminal 413, and may be assembled in a way similar to that described above in connection with FIG. 6. The first pressure sensing terminal 411 is fixedly mounted to the seat bottom plate 2 by fasteners, such as bolts; the force transfer part 412 is connected to the first pressure sensing terminal 411 and extends through the seat bottom plate 2, and is connected to the second pressure sensing terminal 413 at the terminal away from the first pressure sensing terminal 411. The second pressure sensing terminal 413 is connected in a way that it extends beyond the lower surface of the seat bottom plate 2, so that the toilet seat can be supported on the top surface of the toilet seat stand via the second pressure sensing terminal 413 during use, thereby the pressure exerted by the human body on the seat face plate 1 is accurately transferred to the pressure sensor 41, and the body weight of the human body is accurately measured.

Preferably, wires are laid in the cavity between the seat face plate 1 and the seat bottom plate 2, and the signal output terminals of the health detection sensors are electrically connected with the wires in the cavity.

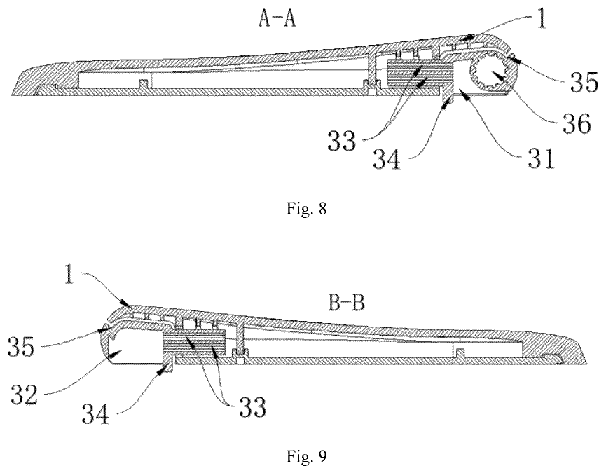

Preferably, as shown in FIGS. 7 to 9, the first connecting part 31 and the second connecting part 32 are provided with legs 34 at the bottom, and the bottoms of the legs 34 are in contact with the toilet seat stand; both the first connecting part 31 and the second connecting part 32 are provided with a mating part 35 at the upper end, and the mating parts 35 are mated with the rear end of the seat face plate 1, and there is a gap between the seat face plate 1 and the mating parts 35 of the first connecting part 31 and the second connecting part 32 when the seat face plate 1 is in the initial state.

The legs 34 are used to present the first connecting part 31 and the second connecting part 32 from being in contact with the toilet seat body when the toilet seat body is in the initial state, so as not to cause stress in the toilet seat body, in order to ensure the accuracy of the detection result. Moreover, with such a structure, the first connecting part 31, the second connecting part 32 and the connected motor can be in separate positions. When the toilet seat is subjected to external force, the seat face plate 1 is deformed downward till it comes into contact with the mating part 35, but the width of the gap is greater than the displacement caused by the downward deformation of the seat face plate 1, thus, the toilet seat is in a separate state when it is deformed downward, and it will not be affected by the first connecting part 31 and the second connecting part 32 at the rear end. In other words, the first connecting part 31 and the second connecting part 32 have no impact on the deformation of the toilet seat during use, thus, the toilet seat in the present disclosure has high measurement accuracy.

Preferably, as shown in FIG. 7, both the first connecting part 31 and the second connecting part 32 are provided with a bushing hole 33, and the wires used for connecting the signal output terminals of the health detection sensors extend out of the cavity through the bushing holes 33; the end of the bushing hole 33 located in the toilet cavity has a protrusion 37, and the seat bottom plate 2 is provided with a limiting groove 21 corresponding to the protrusion 37; the protrusion 37 snap-fitted in the limiting groove 21, and the first connecting part 31 and the second connecting part 32 can be laterally displaced to a limited degree under the limiting effect of the limiting groove 21.

The bushing hole 33 is used for leading the wires out of the cavity of the toilet seat, and the limiting groove 21 plays a limiting role on the first connecting part 31 and the second connecting part 32; there is certain spacing between the bushing hole 33 and the seat bottom plate 2, to prevent the wires from being affected in a case that water exists in the toilet seat; in addition, the limiting groove 21 is provided with a water permeable hole at the bottom, and the protrusion 37 can prevent external water from invading into the cavity, so as to prevent water accumulation in the cavity and ensure a normal working environment for the toilet seat.

Preferably, as shown in FIG. 4, the seat face plate 1 is further provided with a human body sensing sensor 43, and a sensing terminal of the human body sensing sensor 43 is exposed on the upper surface of the seat face plate 1.

Thus, the pressure sensors 41 and the body fat sensors 42 can be woken up only when the human body sensing sensor 43 senses that a human body sits on the toilet seat. In that way, it is unnecessary to keep the detection function in an active state all the time, and, as a result, the energy consumption can be reduced, and the service life of the detection elements can be prolonged.

As a preferred scheme of the connecting mechanism, in actual use, a motor for controlling the rotation of the connecting mechanism is connected between the first connecting part 31 and the second connecting part 32, and the motor is mounted on the surface of the toilet seat stand.

In that way, the motor can control the first connecting part 31 and the second connecting part 32 to rotate, and then drive the toilet seat body to rotate, so that the toilet seat body can be moved to the surface of the toilet seat stand or moved away from the surface of the toilet seat stand. Thus, the toilet seat body can be lifted up or lowered automatically, and the toilet seat is more intelligent.

In actual implementation, the first connecting part 31 and the second connecting part 32 have connecting ends on opposing sides, the connecting end of the first connecting part 31 is a bushing, and the connecting end of the second connecting part 32 is a cogged slot 36; the two ends of the motor are movably connected with the bushing and the cogged slot 36 respectively.

As a preferred scheme of the motor, the motor connected between the first connecting part 31 and the second connecting part 32 may be a health detection controller, which can collect the human health detection data collected by the toilet seat body, and process and transmit the data, while controlling the first connecting part 31 and the second connecting part 32 to rotate; the wires are led out of the bushing hole 33 and then are electrically connected to the health detection controller arranged on the toilet; the signal output terminal of the pressure sensor 41 and the signal output terminal of the human body sensing sensor 43 are connected to signal input terminals of the health detection controller through the wires arranged in the cavity and led out of the cavity; the health detection controller collects and processes the data in the input signals, outputs a result, and transmits the result to the operating terminal for the user to check.

The present disclosure further discloses a detection control method for a toilet seat capable of health detection, which utilizes the toilet seat capable of health detection described above, and comprises the following steps:
step 1: driving the connecting mechanism to rotate the toilet seat body to the surface of the toilet seat stand;
step 2: activating the health detection sensors on the toilet seat body;
step 3: collecting data of human health parameters, by the health detection sensors, when the health detection sensors detect that a human body is seated on the surface of the toilet seat body and has been in contact with the health detection sensors for a preset duration; and
step 4: transmitting the collected data of human health parameters to an operating terminal after the data of human health parameters is collected, thereby completing the collection of human health parameters.

Preferably, in the step 2, the health detection sensors comprise body fat sensors 42 and pressure sensors 41, wherein the body fat sensors 42 are used for detecting body fat parameters, and the pressure sensors 41 are used for detecting body weight.

Preferably, in the step 2, a human body sensing sensor 43 is further provided on the surface of the toilet seat body, and the human body sensing sensor 43 can wake up the pressure sensors 41 and the body fat sensors 42 only when it senses a human body seated on the toilet seat body.

In actual implementation, in the step 4, a health detection controller may be provided and mounted on the surface of the toilet seat; then, a detection instruction may be sent to the health detection controller via the operating terminal, and the health detection controller controls the body fat sensors 42 and the pressure sensors 41 to collect data.

The body weight and body fat detection data collected by the health detection controller can be transmitted to the operating terminal in a wired manner, e.g., through a data cable, or in a wireless manner.

The toilet seat capable of health detection and the detection control method disclosed in the present disclosure have the following technical effects: The toilet seat capable of health detection in the present disclosure has the advantages of a simple overall structure, an integrated design, small overall size, convenient installation, a robust structural connection, stable and reliable operation during use, an attractive appearance, and a sanitary internal structure, etc.; the toilet seat capable of health detection in the present disclosure can detect a variety of physical health indicating parameters, realizes integration of multiple functions, simplifies operation steps, reduces detection difficulties, has higher detection precision and accuracy, and has a promising market value; since the connecting mechanism at the rear end of the body of the toilet seat capable of health detection is separate from the toilet seat and has no impact on the deformation of the toilet seat during use, the toilet seat has high measurement accuracy; the toilet seat capable of health detection in the present disclosure has an integral structure, can be directly used on an ordinary toilet to replace a common toilet cover, is adaptive to a variety of common toilet covers, is conductive to the transformation of common toilets to intelligent ones, and is suitable for wide application in intelligent toilet systems.

It may be understood that the present disclosure is described by means of some embodiments. Those skilled in the art can easily understand that various modifications or equivalent substitutions may be made to the features and embodiments without departing from the spirit and scope of the present disclosure. Under the teaching of the present disclosure, the features and embodiments can be modified to adapt to specific situations and materials without departing from the spirit and scope of the present disclosure. The embodiments described herein are only some embodiments of the present disclosure rather than all possible embodiments of the present disclosure. Generally, the components of the embodiments of the present disclosure described and illustrated in the accompanying drawings herein can be arranged and designed in various different configurations. Therefore, the following detailed description of the embodiments of the present disclosure provided in the accompanying drawings is not intended to limit the scope of the claimed disclosure, but is only intended to explain the selected embodiments of the present disclosure. Therefore, the present disclosure is not limited to the specific embodiments disclosed herein. Based on the embodiments in the present disclosure, those having ordinary skilled in the art can obtain other embodiments without expending any creative labor, but all these embodiments shall be deemed as falling in the scope of protection of the present disclosure.

Paragraph 1. A toilet seat, comprising: a toilet seat body, comprising a seat face plate, a seat bottom plate, and at least one health detection sensor; and a connecting mechanism for rotatably connecting the toilet seat body to a toilet seat stand, wherein the seat face plate and the seat bottom plate are snap-fitted with each other to form a cavity between the seat face plate and the seat bottom plate; wherein when the toilet seat body is rotated to a position of use, the toilet seat body is supported on a top surface of the toilet seat stand by the at least one health detection sensor, and a gap is formed between the toilet seat body and the connecting mechanism so as to allow the toilet seat body to move vertically with respect to the connecting mechanism.

Paragraph 2. The toilet seat of paragraph 1, wherein the at least one health detection sensor each is mounted in the seat face plate, the seat bottom plate, or the cavity.

Paragraph 3. The toilet seat of paragraph 1 or paragraph 2, wherein the at least one health detection sensor comprises a sensing terminal configured to sense a parameter of a human body seated on the seat face plate and a signal output terminal configured to output a sensing signal.

Paragraph 4. The toilet seat of any one of paragraphs 1 to 3, wherein the at least one health detection sensor comprises at least one pressure sensor, wherein the seat bottom plate comprises at least one via-hole, and wherein the pressure sensor comprises a terminal extending beyond a lower surface of the seat bottom plate through the at least one via-hole, configured to support the toilet seat body on the top surface of the toilet seat stand during use.

Paragraph 5. The toilet seat of any one of paragraphs 1 to 4, wherein the at least one health detection sensor comprises a plurality of pressure sensors symmetrically distributed on left and right sides on the horizontal plane of the toilet seat body.

Paragraph 6. The toilet seat of any one of paragraphs 1 to 3, wherein the at least one health detection sensor comprises at least one body fat sensor, wherein the body fat sensor comprises a sensing terminal exposed on the upper surface of the seat face plate, configured to contact with the human body seated on the seat face plate.

Paragraph 7. The toilet seat of paragraph 6, wherein the body fat sensor is configured to release micro-current to the human body so that a closed AC circuit is formed between the human body and the body fat sensor.

Paragraph 8. The toilet seat of paragraph 6 or paragraph 7, wherein the upper surface of the seat face plate comprises a plurality of grooves, and the at least one health detection sensor comprises a plurality of body fat sensors each mounted in one of the grooves respectively.

Paragraph 9. The toilet seat of paragraph 1 or paragraph 2, wherein the at least one health detection sensor comprises a human body sensing sensor mounted in the seat face plate, and said human body sensing sensor comprises a sensing terminal exposed on the upper surface of the seat face plate.

Paragraph 10. The toilet seat of paragraph 1, wherein the connecting mechanism comprises a first connecting part and a second connecting part that are respectively used for connecting to two sides of the rear end of the toilet seat body, and each of the first connecting part and the second connecting part comprises a support leg at the bottom for supporting the connecting mechanism on the top surface of the toilet seat stand when the toilet seat body is rotated to a position of use.

Paragraph 11. The toilet seat of paragraph 10, wherein each of the first connecting part and the second connecting part comprises a connecting head for connecting to the toilet seat body and a pivoting part for connecting to the toilet seat stand, and wherein a mating part corresponding to the rear end of the toilet seat body is formed on the outer periphery of the pivoting part, and said mating part is spaced from the rear end of the toilet seat body to form the gap when the toilet seat body is rotated to the position of use.

Paragraph 12. The toilet seat of paragraph 11, wherein the connecting head comprises a bushing hole for leads to pass there-through, and the leads are electrically connected to the signal output terminal of the at least one health detection sensor.

Paragraph 13. The toilet seat of paragraph 11, wherein the connecting heads of the first connecting part and the second connecting part respectively have protrusions extending towards each other, and the protrusions are snap-fitted in limiting grooves formed in the two sides of the rear end of the toilet seat body.

Paragraph 14. The toilet seat of paragraph 11, wherein the connecting mechanism comprise a motor connected between the respective pivoting parts of the first connection part and the second connection part for controlling the connecting mechanism to rotate with respect to the toilet seat stand.

Paragraph 15. The toilet seat of paragraph 14, wherein the pivoting part of at least one of the first connecting part and the second connecting part has a cogged slot for drivingly connecting to a power output shaft of the motor.

Paragraph 16. An intelligent toilet, comprising a toilet seat stand and the toilet seat of any of paragraphs 1 to 13.

1. A toilet seat, comprising:
a toilet seat body, comprising a seat face plate, a seat bottom plate, and at least one health detection sensor; and
a connecting mechanism for rotatably connecting the toilet seat body to a toilet seat stand,
wherein the seat face plate and the seat bottom plate are snap-fitted with each other to form a cavity between the seat face plate and the seat bottom plate;
wherein when the toilet seat body is rotated to a position of use, the toilet seat body is supported on a top surface of the toilet seat stand by the at least one health detection sensor, and a gap is formed between the toilet seat body and the connecting mechanism allowing the toilet seat body to move vertically with respect to the connecting mechanism.

2. The toilet seat of claim 1, wherein the at least one health detection sensor each is mounted in the seat face plate, the seat bottom plate, or the cavity.

3. The toilet seat of claim 1, wherein the at least one health detection sensor comprises a sensing terminal configured to sense a parameter of a human body seated on the seat face plate and a signal output terminal configured to output a sensing signal.

4. The toilet seat of claim 1, wherein the at least one health detection sensor comprises at least one pressure sensor, wherein the seat bottom plate comprises at least one via-hole, and wherein the pressure sensor comprises a terminal extending beyond a lower surface of the seat bottom plate through the at least one via-hole, configured to support the toilet seat body on the top surface of the toilet seat stand during use.

5. The toilet seat of claim 4, wherein the at least one health detection sensor comprises a plurality of pressure sensors symmetrically distributed on left and right sides on the horizontal plane of the toilet seat body.

6. The toilet seat of claim 1, wherein the at least one health detection sensor comprises at least one body fat sensor, wherein the body fat sensor comprises a sensing terminal exposed on the upper surface of the seat face plate, configured to contact with the human body seated on the seat face plate.

7. The toilet seat of claim 6, wherein the body fat sensor is configured to release micro-current to the human body so that a closed AC circuit is formed between the human body and the body fat sensor.

8. The toilet seat of claim 6, wherein the upper surface of the seat face plate comprises a plurality of grooves, and the at least one health detection sensor comprises a plurality of body fat sensors each mounted in one of the grooves respectively.

9. The toilet seat of claim 1, wherein the at least one health detection sensor comprises a human body sensing sensor mounted in the seat face plate, and said human body sensing sensor comprises a sensing terminal exposed on the upper surface of the seat face plate,

10. The toilet seat of claim 1, wherein the connecting mechanism comprises a first connecting part and a second connecting part that are respectively used for connecting to two sides of the rear end of the toilet seat body, and each of the first connecting part and the second connecting part comprises a support leg at the bottom for supporting the connecting mechanism on the top surface of the toilet seat stand when the toilet seat body is rotated to a position of use.

11. The toilet seat of claim 10, wherein each of the first connecting part and the second connecting part comprises a connecting head for connecting to the toilet seat body and a pivoting part for connecting to the toilet seat stand, and wherein a mating part corresponding to the rear end of the toilet seat body is formed on the outer periphery of the pivoting part, and said mating part is spaced from the rear end of the toilet seat body to form the gap when the toilet seat body is rotated to the position of use.

12. The toilet seat of claim 11, wherein the connecting head comprises a bushing hole for leads to pass there-through, and the leads are electrically connected to the signal output terminal of the at least one health detection sensor.

13. The toilet seat of claim 11, wherein the connecting heads of the first connecting part and the second connecting part respectively have protrusions extending towards each other, and the protrusions are snap-fitted in limiting grooves formed in the two sides of the rear end of the toilet seat body.

14. The toilet seat of claim 11, wherein the connecting mechanism comprise a motor connected between the respective pivoting parts of the first connection part and the second connection part for controlling the connecting mechanism to rotate with respect to the toilet seat stand.

15. The toilet seat of claim 14, wherein the pivoting part of at least one of the first connecting part and the second connecting part has a cogged slot for drivingly connecting to a power output shaft of the motor.

16. An intelligent toilet, comprising a toilet seat stand and the toilet seat of claim 1.
